(19) United States
(12) Plant Patent Application Publication
(10) Pub. No.: US 2014/0041089 P1
(43) Pub. Date: Feb. 6, 2014

(54) Sedum plant named 'Pizzazz'

(50) ;

(71) Applicant: David R. Tristram, Arundel (GB)

(72) Inventor: David R. Tristram, Arundel (GB)

(21) Appl. No.: 13/987,473

(22) Filed: Jul. 29, 2013

Related U.S. Application Data
(60) Provisional application No. 61/742,080, filed on Aug. 1, 2012.

(51) Int. Cl. A01H 5/00

(52) U.S. Cl. PLT/473

(57) ABSTRACT

A new and distinct variety of Sedum named ‘Pizzazz’ that is characterized by clump forming habit, thick mid-green colored leaves, and flowers with long petals which are mid-pink in color in summer, becoming bright cerise at peak of flowering in late summer and early fall, and aging to deep ruby before senescence.

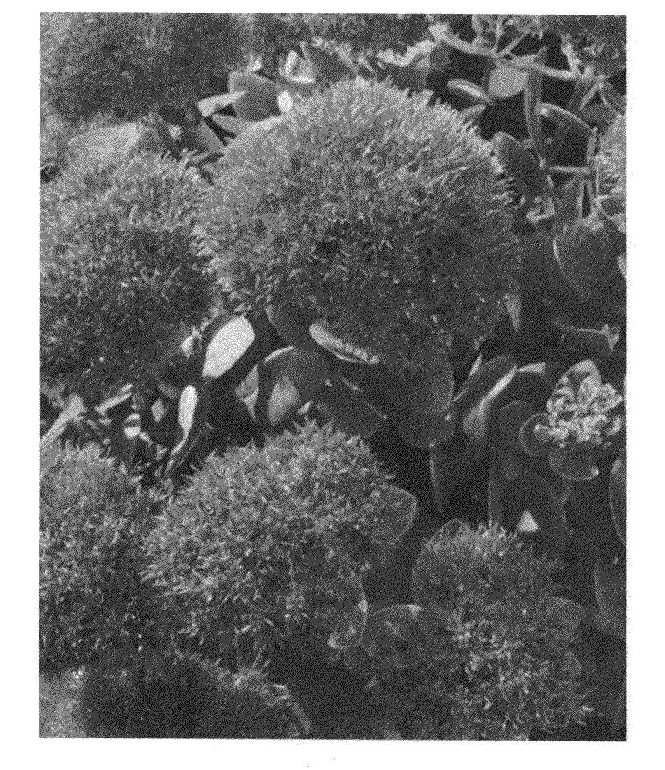

### GENUS AND SPECIES

Sedum spectabile

### VARIETY DENOMINATION

‘Pizzazz’

### CROSS-REFERENCE TO RELATED APPLICATIONS

This application claims the benefit of priority of U.S. Provisional Application No. 61/742,080 filed on Aug. 1, 2012 entitled SEDUM PLANT NAMED ‘PIZZAZZ’.

### BACKGROUND OF THE NEW PLANT

The present invention relates to a new variety of stonecrop grown as an ornamental for use in containers and for growing in the garden border or landscape. The new invention from the family Crassulaceae is known botanically as Sedum spectabile and will be referred to hereinafter as ‘Pizzazz’.

‘Pizzazz’ arose as a seedling which the inventor selected in 2005 from many seedlings raised from the self-fertilization of Sedum ‘Neon’ (unpatented). In comparison with other seedlings, the selected seedling, ‘Pizzazz’ exhibited the brightest flower color and the most compact plant shape.

‘Pizzazz’ was first asexually propagated in 2008 by the inventor in Arundel, West Sussex, United Kingdom. Asexual propagation was accomplished using stem cuttings. Since that time, under careful observation, the distinguishing characteristics of ‘Pizzazz’ have been determined stable and uniform, and to reproduce true to type in successive generations of asexual propagation.

### SUMMARY

‘Pizzazz’ exhibits clumping habit with mid-green foliage. The flowers of ‘Pizzazz’ open mid-pink in color in summer, becoming bright cerise at peak flowering in late summer and early fall. During peak flowering, the stamens of ‘Pizzazz’ are prominently exserted and brightly cerise in color. Plants of ‘Pizzazz’ achieve 35.0 cm to 45.0 cm in height and 30.0 cm to 40.0 cm in width after one year of growth, and reach 45.0 cm in height and 45.0 cm in width at maturity. Cultural requirements include free-draining soil, full sun, and minimal water. ‘Pizzazz’ is susceptible to snails, slugs, and mildew. Spraying is recommended in areas of high humidity to prevent mildew, and use of snail bait is recommended to prevent damage from snails and slugs. ‘Pizzazz’ is hardy in USDA Zone 5.

The following traits have been repeatedly observed and represent the distinguishing characteristics of the new Sedum variety named ‘Pizzazz’. These traits in combination distinguish ‘Pizzazz’ from all other existing varieties of Sedum known to the inventor. ‘Pizzazz’ has not been tested under all possible conditions. Phenotypic differences may be observed with variations in environmental, climatic, and cultural conditions.

1. Plants of ‘Pizzazz’ exhibit a compact clump-forming growth habit.

2. The foliage of ‘Pizzazz’ is mid-green in color.

3. During peak flowering, the inflorescence of ‘Pizzazz’ is bright cerise in color.

4. During peak flowering, the stamens of ‘Pizzazz’ are prominently exserted and are bright cerise in color.

5. The flowers of ‘Pizzazz’ open mid-pink in summer, becoming bright cerise at peak of flowering in late summer, aging to deep ruby before senescence.

6. At one year of age, plants of ‘Pizzazz’ are 35.0 cm to 45.0 cm in height and 30.0 cm to 40.0 cm in width.

7. At maturity, plants of ‘Pizzazz’ are 45.0 cm in height and width.

8. ‘Pizzazz’ blooms from summer to fall.

9. ‘Pizzazz’ is hardy to USDA Zone 5.

### COMPARISON WITH PARENTAL LINE AND KNOWN VARIETY

‘Pizzazz’ is unique and distinguishable from any other Sedum known to the inventor. The closest comparison plant is Sedum ‘Autumn Joy’ (unpatented). Whereas the flowers of ‘Autumn Joy’ open dark pink and aging to brick-red, the flowers of ‘Pizzazz’ flowers open mid-pink in color in summer, becoming bright cerise at the peak of flowering in late summer and early fall, and aging to deep ruby. In addition, the growth habit of ‘Pizzazz’ is more compact and shorter than ‘Autumn Joy’.

When compared with the parental variety ‘Neon’, ‘Pizzazz” exhibits brighter flower colors and a more compact habit.

### BRIEF DESCRIPTION OF THE PHOTOGRAPH

The accompanying photograph illustrates a close-up of ‘Pizzazz’ showing the colors of its foliage and flowers as true as it is reasonably possible to obtain in colored reproductions of this type. The photograph depicts the typical and characteristic bright cerise inflorescences of ‘Pizzazz’ when the individual flowers in the inflorescence are semi-open to fully-open. The plants in this photograph are approximately one-year-old and have been grown outdoors in 3-liter containers in Arundel, West Sussex, United Kingdom. The photograph was taken in September 2010.

### BOTANICAL DESCRIPTION OF THE PLANT

The following is a detailed botanical description of the new Sedum variety named ‘Pizzazz’. Observations, measurements, values and comparisons were collected in Santa Barbara, Calif., in July 2013 from 18-month-old plants grown outdoors in the border soil. Color determinations are made in accordance with the 2007 Royal Horticultural Society Colour Chart from London, England, except where general color terms of ordinary dictionary significance are used. The growing requirements of the new variety ‘Pizzazz’ are similar to the species.
Botanical classification:
   Family.—Crassulaceae.
   Genus and species.—Sedum spectabile.
   Denomination.—‘Pizzazz’.
   Common name.—Stonecrop.
Parentage: Selected seedling raised from the self-fertilization of Sedum ‘Neon’ (unpatented)
Plant:
   Habit.—Clump forming.
   Commercial category.—Succulent.
   Use.—Container and border.
   Suggested commercial container size.—2-liter container.
   Propagation method.—Stem cuttings.
   Rooting system.—Fine and fibrous.
   Vigor.—Moderate.
   Crop time (range).—8 to 16 weeks from a cutting to a 5.0 cm liner pot and 16 to 24 weeks from a liner pot a finished 2-litre container plant in flower.
   Plant dimensions (first year).—35.0 cm to 45.0 cm in height and 30.0 cm to 40.0 cm in width.
   Plant dimensions (at maturity).—45.0 cm in height and width.
   Cultural requirements.—Grow in free-draining soil and full sun, with minimal water.
   Pest and disease resistance.—None known to the inventor.
   Pest and disease susceptibility.—Mildew, snails and slugs.
   Hardiness.—USDA Zone 5.
Stem:
   Branching habit.—Rosette.
   Trunk height.—8.0 cm to 10.0 cm.
   Trunk diameter.—3.0 cm to 4.0 cm.
   Trunk color.—145A.
   Stem surface.—Glabrous.
   Stem length.—20.0 cm to 30.0 cm.
   Stem diameter.—1.0 cm to 1.3 cm.
   Stem color.—145A.
   Stem strength.—Strong.
   Stem appearance.—Semi-glossy.
   Stem quantity (average).—16.
   Internode distance (average).—0.50 cm.
Foliage:
   Leaf quantity (range).—35 to 45 per stem.
   Leaf attachment.—Sessile.
   Leaf arrangement.—Alternate.
   Leaf texture.—Rubbery and thick.
   Leaf division.—Simple.
   Leaf margin.—Entire.
   Leaf surface (abaxial and adaxial).—Glabrous.
   Leaf shape.—Elliptic.
   Leaf length (average).—6.0 cm.
   Leaf width (average).—3.0 cm.
   Leaf color (both surfaces).—143C with cream pink coloration to leaf edges in hot dry conditions.
   Leaf apex (range).—Acute to rounded.
   Leaf base.—Truncate.
   Venation.—Pinnate.
   Vein color (abaxial and adaxial surfaces).—143C.
Inflorescence:
   Inflorescence type.—Flat topped cyme.
   Blooming season.—Summer to fall.
   Inflorescence quantity (average).—16.
   Inflorescence dimensions (fully expanded).—5.0 cm to 10.0 cm in height, 12.0 cm to 18.0 cm in width.
   Flower quantity per inflorescence.—Very numerous; approximately 250 to 300.
   Flower aspect.—Facing upward.
   Persistent or self-cleaning.—Persistent.
   Flower dimensions.—6.0 mm in depth and 10.0 mm in diameter.
Bud:
   Bud shape.—Ovoid.
   Bud dimensions.—3.0 mm in length and 2.0 mm in width.
   Bud surface.—Glabrous.
   Bud apex.—Acute.
   Bud base.—Rounded.
   Bud color.—N155C.
Petals:
   Petal quantity.—5 in number.
   Petal color.—Ranges between N57A and N57B.
   Petal shape.—Oval.
   Petal apex.—Acute.
   Petal base, fused or unfused.—Unfused.
   Petal margin.—Entire.
   Petal dimensions.—7.0 mm to 8.0 mm in length and 2.0 mm in width.
   Petal surface texture (abaxial and adaxial).—Glabrous.
Sepals:
   Sepal quantity.—5 in number.
   Sepal shape.—Oval.
   Sepal color.—N144A.
   Sepal apex.—Acute.
   Sepal base, fused or unfused.—Unfused.
   Sepal margin.—Entire.
   Sepal dimensions.—1.0 mm in length and 0.50 mm in width.
   Sepal surface texture (abaxial and adaxial).—Glabrous.
Peduncle:
   Peduncle shape.—Cylindrical.
   Peduncle color.—N144A.
   Peduncle surface texture.—Glabrous.
   Peduncle dimensions.—2.50 cm in length and 3.0 mm in diameter.
Pedicel:
   Pedicel shape.—Cylindrical.
   Pedicel color.—N144A.
   Pedicel surface texture.—Glabrous.
   Pedicel dimensions.—0.50 cm in length and 1.0 mm in width.
   Flower fragrance.—None observed.
Reproductive organs:
   Stamen.—10 in number, 5 short, approximately 2.0 mm in length and attached to petal base; 5 long, approximately 6.0 mm to 10.0 mm in length, alternating with petals.
   Stamen color.—N57A.
   Anthers.—Shape ellipsoid, 1.5 mm in length, 1.0 mm in width, color 60C.
Seed: Fine, ovoid, less than 0.5 mm in length.

I claim:

1. A new and distinct cultivar of Sedum plant named ‘Pizzazz’ as described and illustrated herein.
